(19) United States
(12) Patent Application Publication
(10) Pub. No.: US 2009/0050145 A1
(43) Pub. Date: Feb. 26, 2009

(54) NASAL CATHETER WITH INTERNAL AND EXTERNAL AIR FLOWS

(75) Inventors: Bertrand De Silva, Yorba Linda, CA (US); Brian E. McIntee, Trabuco Canyon, CA (US); Michael Schultz, Santa Cruz, CA (US)

Correspondence Address: FISH & ASSOCIATES, PC;ROBERT D. FISH, 2603 Main Street, Suite 1050, Irvine, CA, 92614-6232, US

(73) Assignee: RESPIRION INCORPORATED, Trabuco Canyon, CA (US)

(21) Appl. No.: 12/261,404

(22) Filed: Oct. 30, 2008

Related U.S. Application Data
(63) Continuation-in-part of application No. 11/561,498, filed on Nov. 20, 2006.
(60) Provisional application No. 60/823,910, filed on Aug. 30, 2006.

(51) Int. Cl. A61M 16/00; A61M 29/00

(52) U.S. Cl. 128/200.24; 606/199

(57) ABSTRACT

A nasally introduced catheter (nasal catheter) has a portion in which a user's breath (breathing air) air travels internally and a portion in which the breath travels externally. The portion in which air flow travels internally preferably includes a mesh or a whisk-like portion that expands and contracts, and the portion in which air flow travels externally preferably comprises a proximal tubular element having a sufficiently small cross-sectional area that at least 50% of the breathing air flows externally to the proximal portion. A sheath can optionally be positioned about the distal portion, and pulled back along the proximal portion to allow the distal portion to expand. In other aspects users can anchor the proximal and distal portions relative to the sheath using a nose clip or by removably coupling an extension arm to proximal portion.

This application is a continuation-in-part of claims priority to pending U.S. utility application Ser. No. 11/561498 filed Nov. 1, 2006, which claims priority to provisional application Ser. No. 60/823910 filed Aug. 30, 2006, and further claims priority to provisional application Ser. No. 60/983667 filed Oct. 30, 2007.

### FIELD OF THE INVENTION

The field of the invention is nasal catheters (class 128/207.18).

### Background

There are several diseases, conditions, and situations in which it is desirable to artificially maintain patency of the nasal passageway and pharynx.

The simplest solution is to insert a tube of sufficient length and internal diameter through a nostril and into to the nasal passageway. The lumen of the tube then provides the airway. With minor modifications, that approach was adopted in Dunlap 2006/0048775 (March 2006). Dunlap and all other referenced extrinsic materials are incorporated herein by reference in their entirety. Where a definition or use of a term in an incorporated reference is inconsistent or contrary to the definition of that term provided herein, the definition of that term provided herein applies and the definition of that term in the reference does not apply. Unless a contrary intent is apparent from the context, all ranges recited herein are inclusive of their endpoints, and open-ended ranges should be interpreted to include only commercially practical values.

The main problem with the Dunlap approach is that the inner diameter of the tube must be at least 5 mm, and more preferably at least 10 mm. Such tubes are very uncomfortable for the users. An alternative approach is typified by Dubrul 2004/0020492 (February 2004) in which a wire mesh is inserted in a compressed configuration, and then expanded along its entire length. Dubrul's approach might be somewhat better tolerated than Dunlap because the walls comprise a mesh as opposed to a solid barrier. But Dubrul still contemplates a fully deployed catheter that comprises a large tube over substantially the entire length of the device inside the user.

There is another, very serious problem with Dubrul, namely lack of enablement as to how the sheath 4 is to be slid relative the mesh (support element) 6 and the proximal end 10. There is no disclosure at all as to what the user can hold onto while pulling or pushing on the sheath 4. When fully inserted, the only portion that could be held by the user is proximal end 10, and that proximal end 10 would be inside the sheath 4 during at least some portion of the sheath travel.

Thus, there is still a need for a practical nasal catheter, one that has an expandable portion and a more tolerable cross-section for a portion that is either not expandable or at least less expandable.

### SUMMARY OF THE INVENTION

The present invention provides apparatus, systems and methods in which a nasally introduced catheter (nasal catheter) has a portion in which a user's breath (breathing air) air travels internally and a portion in which the breath travels externally.

In preferred embodiments, the portion in which airflow travels internally comprises a distal portion of the device, and can for example, include a mesh or a whisk-like portion that expands and contracts. Also in preferred embodiments, the portion in which air flow travels externally comprises a proximal tubular element having a sufficiently small cross-sectional area that at least 50% of the breathing air flows externally to the proximal portion.

The distal, expandable portion preferably has a length of between 5 cm and 13 cm when in an expanded configuration, and between about 10 cm and 16 cm in a contracted or other non-expanded configuration. In typical contemplated configurations, the fully expanded configuration is no more than 30% shorter than the fully contracted configuration. Viewed from another perspective, the distal, expandable portion preferably has a maximum cross-section of no more than 18 mm² when in a non-expanded configuration and a maximum cross-section of no less than 50 mm² when in the expanded configuration.

The proximal portion can comprise a single wire, braided or twisted wires, or even an inner member that is co-axial and slidable within an outer member. In that later embodiment, the inner member preferably extends to a nub or other functionally distal end of the distal portion. Pulling the inner member proximally pulls on the nub, and thereby bends wires in the distal portion from a substantially parallel configuration to a substantially whisk-like bulbous configuration.

Nose clips or other stops are also contemplated. Preferred stops extend from the proximal portion and bend around one of the user's nostrils, at least sufficiently to prevent the end of the proximal portion from entering the nostril. In some contemplated variations, a nose clip is functionally coupled to the distal end of the distal portion, such that operation of the nose clip expands or reverses expansion of the distal portion,

In some embodiments a sheath can be positioned about the distal portion, and pulled back along the proximal portion to allow the distal portion to expand. Pushing the sheath back over the distal portion causes reversal of the expansion. It is contemplated that users can anchor the proximal and distal portions relative to the sheath in any suitable manner, including: (1) using the nose clip as the anchor, and running the sheath along the outside of the proximal portion via a slit along the side of the sheath; and alternatively (2) removably coupling a extension arm to proximal portion, and using the extension arm as the anchor.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a perspective side view of a preferred embodiment.

FIG. 2 is a perspective side view of an alternative embodiment in which the proximal portion has an inner member slidable against an outer member.

FIG. 3 is a perspective side view of an alternative preferred embodiment having a removable extension arm.

FIG. 4A is a perspective side view of a preferred embodiment in which the wires of the distal portion are parallel.

FIG. 4B is a perspective side view of the embodiment of FIG. 4A, where wires of the distal portion are distorted into a whisk-type configuration.

FIG. 5A-5C are a perspective side views of a distal portion of the embodiment of FIGS. 4A and 4B, in varying stages of deployment.

### DETAILED DESCRIPTION

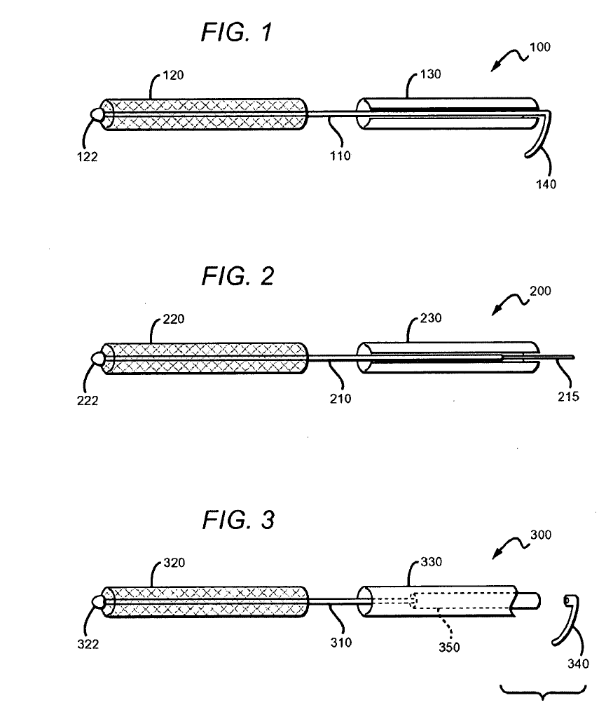

In FIG. 1 a nasal catheter 100 generally includes a proximal portion 110, a distal portion 120 with a nub 122 at the end, a removable sheath 130, and a nose clip 140.

The proximal portion 110 is a wire of satisfactory length, which is currently contemplated to include lengths sufficient to reach from the external nares to the posterior choanae. Currently preferred lengths are 5-10 cm, with this and all ranges being inclusive of their endpoints unless the context clearly dictates the contrary. Wire 110 can be made of any suitable material or materials, provided they are sufficiently flexible to be inserted in the nasal passageway of a typical human without causing substantial damage to the tissues, but also having sufficient column strength to enable proper insertion of the distal portion. The wire 110 should also be non-kinkable under ordinary operating conditions. Wires 110 can be mono- or poly-filamentous, and in the later case can be twisted, wound, or woven. At present, the most preferred wires 110 are wound, comprise a nitinol or other shape-memory metal or alloy, and have an outside diameter of less than 6 Fr. (2 mm), and are coated with a terephthalate or other suitable bio-compatible polymer to reduce friction.

The distal portion 120 can have any suitable dimensions, including for example a length of between 7 cm and 13 cm when in an expanded configuration, and more preferably between about 9 and 11 cm. Preferred distal portions also have a maximum cross-section of no more than 10 mm² when in the non-expanded configuration, and no less than 75 mm² when in the expanded configuration. Both length and diameter dimensions are derived from typical adult anatomy, and other sizes are also contemplated, such as to accommodate children and very large adults.

Distal portion 120 can advantageously comprise the same material(s) and coating(s) as the proximal portion, but could be also be quite different. Where the distal portion comprises a mesh, it is important that the mesh defines spacing sufficient to accommodate both expected airflow and ordinary fluid drainage. In a currently preferred embodiment the spacing is approximately a square measuring about 2 mm on a side in the expanded configuration. In the expanded state the mesh would need to have sufficient radial force to part the pharyngeal tissue and provide a patent physiologic airway. But at the same time the radial force should advantageously be less than the tissue perfusion pressure to ensure adequate tissue oxygenation.

It is important that the distal portion 120 is biased to the expanded configuration, but that the expansion can be manually reversed to a compressed configuration by sliding the sheath 130 over the expanded distal portion with a reasonable amount of effort. The distal portion 120 has a length in an expanded configuration that is at least 50% less than in a contracted configuration, more preferably at least 20% and most preferably at least 25%.

Nub 122 is optional. One could alternatively leave the distal portion open at its distal end. In that case the end is preferably woven back or otherwise adapted to reduce injury to adjacent tissues. Where nub 122 is present, it is preferably made of a plastic or other relatively soft material, and is preferably bull-nosed to allow for convenient insertion.

It is also considered advantageous that the nub can be withdrawn into the mesh so that it does not irritate or tickle the vocal chords. This could be accomplished by inherent design of the mesh, so that when the mesh is expanded the nub is automatically pulled back. In the embodiment of FIG. 2, a nasal catheter 200 generally includes a proximal portion 210, a distal portion 220 with a nub 222 at the end, a removable sheath 230. But in this case the proximal portion 210 has an outer member having a lumen through which an inner member 215 slides. Inner member 215 is coupled to nub 222, and can be used to invaginate nub 222 back into the lumen of the mesh of distal portion 220. In this instance nub 222 should be considered a functionally distal end of the distal portion 220.

Removable sheath 130, 230 is preferably similar to cardiovascular guide catheter in that it needs both considerable column strength and lateral bending flexibility, with a relatively thin wall. This can readily be accomplished using a plastic embedded with metal strands. At its distal end, the sheath 130, 230 is preferably tapered to fit flush with the proximal end of the nub 122, 222. This expected to facilitate atraumatic insertion. Sheaths 130, 230 can be any convenient length, because it will likely not be left in the body.

Sheaths 130, 230 can be split, so that they can be placed over the protrusion of the proximal portion out of the nose, which is likely continuous with the nose clip 140. See FIG. 1. At the proximal end of the sheath 130, 230, one could have a handle, grip or other protuberance that facilitates gripping and manipulating of the sheath 130, 230 by the user or his health care provider.

In an alternative shown in FIG. 3, a nasal catheter 300 generally includes a proximal portion 310, a distal portion 320 with a nub 322 at the end, a removable sheath 330, and a nose clip 340. Here, however, the nose clip 340 is removably attached to the proximal portion 310 (such as for example with a snap or a threaded connection) and there is an extension arm 350 that is removably coupled to the proximal portion 310. In practice one would insert the catheter 300 by sliding the sheath 330 over the distal portion 320, inserting the catheter 300 into the nose of a user (not shown) an appropriate distance, connect the extension arm 350 to the proximal end of the proximal portion 310, withdrawing the sheath 330 over the proximal portion 310 and then over the extension arm 350, detach the extension arm 350 from the proximal portion 310, and finally attach the nose clip 340 to the proximal portion 310. When removing the catheter 300, one reverses the steps above. Extension arm 350 can, of course, be made of any suitable materials and have any suitable length.

Nose clip 140, 340, 440 primarily functions to prevent the proximal portion 110, 210, 310 from sliding further than intended into the nasal cavity, and in extreme situations, advancing to a point that it cannot be easily retrieved. In that light the term nose clip should be construed euphemistically as including any sort of position retaining device that can prevent such untoward events. Thus, for example, a nose clip can be a simple bent wire 140 such as that shown in FIG. 1, or a combination plastic and metal piece such as that shown in FIG. 3. In still further alternative embodiments (not shown), the retaining device could be a flared segment, a band that can be positioned about the head, and so forth.

Another purpose for the nose clip is to act as a positioning guide, indicating that the device is appropriately inserted. The nose clip can also be rotated clock-wise or counter-clockwise by a few degrees, which would also tend to rotate the entire length of the device, and thereby reducing potential for repetitive contact injury and areas of ischemic tissue damage. Users should in fact be encouraged to rotate the nose clip by a few degrees each day.

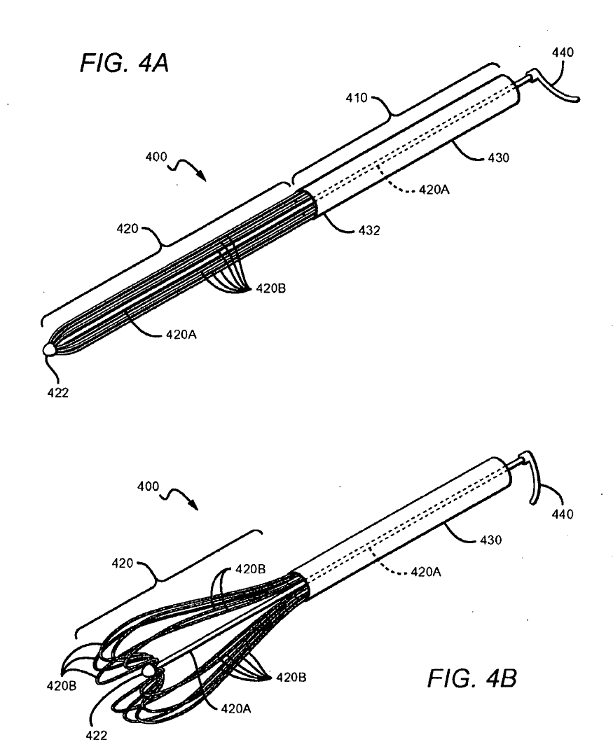

In FIGS. 4A and 4B, a nasal catheter 400 generally includes a proximal portion 410, a distal portion 420 with a collection of wires 420A, 420B terminating at a nub 422, a sheath 430, and a nose clip 440.

One of the wires 420A is slidable with respect to the sheath 430, and the other wires 420B are not slidable with respected to the sheath. In that arrangement, the distal and proximal portions 410, 420 can be inserted through the nose, with the distal portion 420 being in a contracted configuration. Retracting the slidable wire actuates the device by distorting the orientation of the remaining wires 420B to form a whisk-shaped distribution shown in FIG. 4B. The motion is similar to that found in the guidewire system of Cordis™ Angioguard XP™, except that in the Cordis device the guidewires go flush by pulling the ends apart, and in the embodiments of FIGS. 4A, 4B, the wires expand to the whisk-shaped distribution by bringing the ends towards one another (i.e. bringing the nub 422 proximal).

Catheter 400 preferably has between 8-20 wires 420B. It is currently contemplated that operation at the lower end of the range and below would tend to allow excessive tissue prolapse between the wires, and operation at the higher end of that range and above would tend to have inadequate radial separation and cause inspisated mucus buildup.

The wires 420B can extend proximally any suitable distance. They could, for example, terminate at or approximately at the distal end 432 of the sheath 430 (as shown in the figures), pass all the way to the proximal end of the sheath 430, or terminate anywhere in between.

It should also be appreciated that wires 420B need not have a round cross-section. They could, for example, be flattened into ribbons, or have ovoid cross-sections.

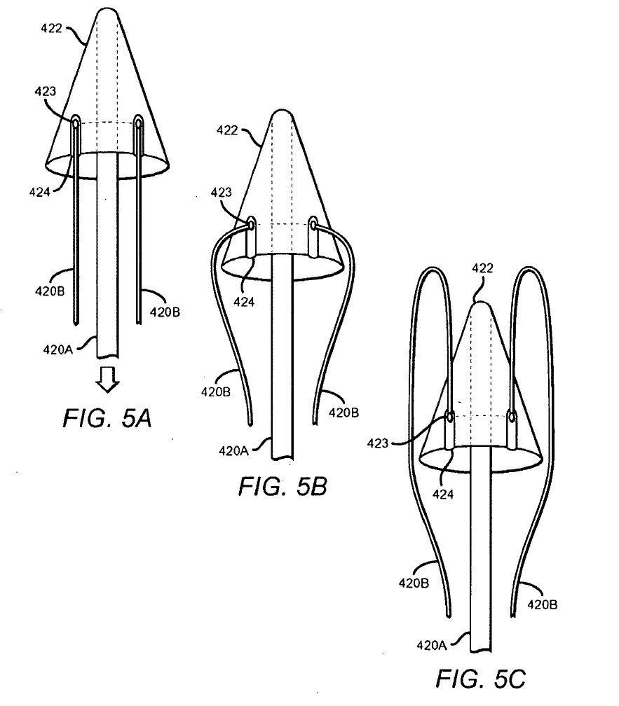

In FIGS. 5A, 5B, 5C the nub 422 is coupled to the wires 420B at pivot points 423. This is intended to reduce breakage of the wires 420B and increase the lifespan of the device 400. The nub 422 also preferably includes grooves 424 that allow the wires to become flush with the surface of the nub 422, which is expected to facilitate insertion. Those of ordinary skill in the art will appreciate that the FIGS. 5A, 5B, 5C only depict two wires 420B for the sake of clarity. A practical embodiment would likely have eight or more.

Thus, specific embodiments and applications of nasal catheters have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A device for maintaining patency of a pharyngeal passageway in a user, comprising:
a distal portion that upon deployment defines an expanded space inside of which breathing air flows; and
a proximal portion that upon deployment has a sufficiently small cross-sectional area that at least 50% of the breathing air flows externally to the proximal portion.

2. The device of claim 1, wherein the distal portion comprises a plurality of substantially parallel wires in a non-expanded configuration.

3. The device of claim 1, wherein the plurality of parallel wires has a whisk orientation in an expanded configuration.

4. The device of claim 1, wherein the distal portion comprises a mesh.

5. The device of claim 1, wherein the distal portion has a length of between 5 cm and 13 cm when in an expanded configuration.

6. The device of claim 1, wherein the distal portion has a maximum cross-section of no more than 18 mm² when in a non-expanded configuration.

7. The device of claim 1, wherein the distal portion has a maximum cross-section of no less than 50 mm² when in the expanded configuration.

8. The device of claim 1, wherein the distal portion has a length in an expanded configuration that is no more than 30% less than in a contracted configuration.

9. The device of claim 1, wherein the proximal portion has an inner member co-axial with an outer member.

10. The device of claim 9, wherein the inner member extends to the distal portion.

11. The device of claim 9, wherein the inner member extends to a functionally distal end of the distal portion.

12. The device of claim 1, further comprising a stop extending from the proximal portion that prevents an end of the proximal portion from entering the nostril.

13. The device of claim 1, further comprising an actuating nose clip.

14. The device of claim 1, further comprising a sheath that is slidable along the proximal portion.

15. The device of claim 14, wherein the sheath is open along at least a portion of its length.

16. The device of claim 1, further comprising an extension arm that is removably coupleable to the proximal portion.
